(19) United States
(12) Patent Application Publication
(10) Pub. No.: US 2016/0095800 A1
(43) Pub. Date: Apr. 7, 2016

(54) POLYURETHANE ELASTOMER DENTAL APPLIANCE

(71) Applicants: Li Luo Skelton, San Antonio, TX (US); Danielle Ruggiero, Canton, GA (US)

(72) Inventors: Li Luo Skelton, San Antonio, TX (US); Danielle Ruggiero, Canton, GA (US)

(21) Appl. No.: 14/723,499

(22) Filed: May 28, 2015

Related U.S. Application Data
(60) Provisional application No. 62/059,281, filed on Oct. 3, 2014.

(51) Int. Cl. A61K 6/09; A61C 13/087; A61C 13/08; A61C 1/08; A61C 13/00

(52) U.S. Cl. CPC A61K 6/09; A61C 1/084; A61C 13/0022; A61C 13/082; A61C 13/087

(57) ABSTRACT

A dental jacket made of a hard, solvent free, substantially non-hydrophilic polyurethane elastomer. The jacket elastomer has a hardness of not less than about Shore D65. A polyester or polyether polyol with a functionality greater than two and a molecular weight greater than one hundred is reacted with an isocyanate prepolymer prepared from one or more branched polyester or polyether polyols with a functionality greater than two; and then blending prepolymer and first polyester or polyether polyol with nanoparticles. In one embodiment, the polypropylene glycol triether and the isocyanate prepolymer comprises dicyclohexylmethane 4,4′-diisocyanate.

### BACKGROUND OF THE INVENTION

This application claims priority to co-pending U.S. Provisional Patent Application Ser. No. 62/059,281, filed Oct. 3, 2014, and U.S. Utility patent application Ser. No. 14/602,343, both of which are incorporated herein for all purposes

This invention relates to a dental jacket made of a hard, shock-absorbing polyurethane elastomer. More particularly, the dental jacket is made of an elastomer having a hardness of not less than about Shore D65 and is made by reacting a polyester or polyether polyol with an isocyanate prepolymer and blending them with nanoparticles and colorants as needed.

Dental appliances, as that term is used herein includes dental jackets (customized or pre-made) full or partial dentures, full or partial implant dentures, fixed or removable implant bridges, implant crowns, Maryland bridges, surgical guides, milling blocks, and individual false teeth that may be fixed or removable replacements for teeth. Tooth replacement becomes necessary when there is single or multiple tooth loss, full arch and/or full mouth tooth loss or roots have been irreparably damaged, and the tooth has been lost or must be removed. These losses or damage may be due to many causes, including but not limited to, genetic factors; economic difficulties; poor education; neglected and poor diet and hygiene; systemic related tooth loss (such as autoimmune disease); aging and excess wear due to stress; advanced periodontal disease and caries; and failing crowns, bridges, or other prostheses.

Dentists have long known that a missing permanent tooth should always be replaced or else the teeth on either side of the space gradually tilt toward the gap, and the teeth in the opposite jaw begin to move toward the space. Dentists have also known that missing teeth may result in not only physical damage, but also mental and psychological damage.

Generally speaking, the adverse effects of tooth and teeth loss may be summarized as including bone loss; loss of chewing power and chewing function; loss of facial esthetic; loss of the stability of dentition for single or multiple teeth loss; loss of occlusion stability thereby resulting in TMJ issues; loss of phonics resulting in speech problems; loss of self- confidence; poor digestion and nutrition; and instability of the entire spine and pelvis complex due to missing teeth causing occlusal problems.

There are several standard forms of tooth replacement in modern dentistry. Dentures are false removable teeth that are relatively quick and easy to fabricate. They may be partial, where only one or a few teeth are replaced; or they may be complete, where all the teeth are missing in the jaw and are replaced.

A partial denture holds false teeth on a plastic or metal framework. The framework is removable, and is designed to fit around the adjacent teeth. It may cover part of the gum tissue or roof of the mouth. There may also be hooks and rests to help hold or support the framework. Sometimes these hooks or rests are visible when smiling. Dentures are normally removed for nightly cleaning and to allow the gum tissue to breathe.

Another form of tooth replacement is the bridge. A bridge uses natural teeth adjacent to an empty space to support a false tooth/teeth there between. The natural teeth usually need to be shaved or contoured so that a crown (cap) may be placed over the top of the natural teeth. These are called abutment teeth. The crowns on each tooth support the false too/teeth there between, known as the pontic tooth/teeth.

Dental implants may also replace natural teeth. Implants are usually composed of two major components: the implant which acts as an anchor in the jaw; and the crown which is the tooth part visible in the mouth. Implants are excellent options for tooth replacements as they do not affect adjacent teeth and yet look, feel, and function as natural teeth do.

An overdenture may be used when the natural root of a tooth is preserved to prevent or delay bone loss that occurs when the entire tooth is removed. The dental professional bonds metal attachments to the root of the tooth and then snaps an overdenture into them. The remaining roots actually provide increased sensation to the wearer of the overdenture and the dentures feel more similar to natural teeth.

Some patients do not like the feeling of a denture on the roof of the mouth or palate. These patients experience a gagging sensation and find swallowing (and even speaking) difficult with a full palate denture. A palate-less, snap on denture is one solution to this situation. In order for a palate-less snap on denture to function properly, it must be snapped into 4-5 implants depending on the size of the patient's arch. Plate-less snap on dentures are a favorite among patients because of the higher comfort level associated with very secure and stable teeth.

The All-on-4® treatment concept provides edentulous and soon-to-be edentulous patients with a fixed full-arch prosthesis on four implants on the day of the surgery.

A new full mouth implant treatment has been developed by one of the inventors herein which provides and instant smile and function make-over. This unique treatment features utilization of the present inventive polyurethane elastomer and incorporates a holistic and lifelong oriented support program. The full mouth implant uses six implants to support a full arch, detachable, fixed nano-polymer bridge.

Historically, a variety of materials have been used to replace lost teeth. Animal teeth and pieces of bone were among the earliest of these primitive replacement materials. In the last few hundred years, artificial teeth have been fashioned from natural substances such as ivory, porcelain, and even platinum. These comparatively crude prototypes of earlier times were carved or forged by hand in an attempt to mimic the appearance and function of natural teeth.

Modern technology has offered considerable advances in the materials used to make artificial teeth and improved techniques for affixing them in the mouth. Synthetic plastic resins and lightweight metal alloys have made teeth more durable and natural looking. Better design has resulted in dentures that provide more comfortable and efficient chewing.

Most artificial teeth are made from high quality acrylic resins bonded to an acrylic base, which make them stronger and more attractive than was once possible. The acrylic resins are relatively wear-resistant, and teeth made from these materials are expected to last between five and eight years. The main problems with such artificial teeth occur with the acrylic base dental appliance, especially the implant support prosthesis. The tooth/teeth/pop out from the base and there is cracking and chipping of the teeth. Further the appliance may easily break causing considerable injury to the wearer.

A flexible-base nylon dental appliance provides a strong and light, metal free partial denture, but its major disadvantage is its inability to be relined or added onto. Such flexible-base nylon are difficult to manufacture and require the use of special thermal injection machinery.

Porcelain is also used as a tooth material because it looks more like natural tooth enamel. Porcelain is used particularly for upper front teeth, which are the most visible. However, the pressure of biting and chewing with porcelain teeth can wear away and damage natural teeth. Further, porcelain provides no shock absorption function. This places more bite forces on other components of the dental appliance, especially underneath supported bone. Even with high quality acrylics and porcelains used today, cracking and chipping of the teeth continues to be a significant problem. With dental appliances made of these current compositions, the tremendous forces created by the wearer during mastication may be transferred through the appliance to the muscles and bone. The appliance may not crack, chip, or break before causing considerable injury to the wearer. There continues to be a need for a dental material which is hard and strong but with flexibility to absorb and yield without cracking and breaking.

Every individual's mouth is different, and each dental appliance must be custom designed to individually fit for functionality and still be cosmetically effective. The latest methodology used in denture design, known as dentogenics, has developed standards for designing teeth to fit specific smile lines, mouth shapes, and personalities. These standards are based on such factors as mouth size and shape, skull size, age, sex, skin color, and hair color. For example, through proper appliance design, patients can be given a younger smile by simply making teeth longer than they normally would be at that patient's age. This rejuvenation effect is possible because a person's teeth wear down over time; slightly increasing the length of the front teeth can create a more youthful appearance.

The unique combination of hardness, flexural strength, and thermal elastomer properties of the present inventive composition result in a hard, shock-absorbing polyurethane elastomer which solves the problems associated with present materials and appliances. The present composition allows for an ideal, moldable, premade dental jacket which maintains the principle of dentogenics, but also provides a clinic with a simple procedure for rapid production, allowing for one-visit treatment. These developments have been made possible by the improvements to the material used to make the dental appliances.

### SUMMARY OF THE INVENTION

The present invention is a dental jacket made of a hard, solvent free, substantially non-hydrophilic polyurethane elastomer having a hardness of not less than about Shore D65 by reacting a polyester polyol with a functionality greater than two and a molecular weight greater than one hundred with an isocyanate prepolymer (either aliphatic or aromatic) prepared from one or more branched polyester or polyether polyols with a functionality greater than two; blending said prepolymer and polyester or polyether polyol with nanoparticles. Using the elastomer various dental appliances, including customized and premade dental jackets, full and partial dentures, full and partial implant bridges, implant crowns, Maryland bridges, surgical guides, millable dental blocks, and replacement teeth may be fabricated.

### BRIEF DESCRIPTION OF THE DRAWINGS

There are no drawings in this disclosure.

### DETAILED DESCRIPTION OF THE PREFERRED EMBODIMENT

The following is a brief discussion of the manufacturing process for making a dental appliance. The process may be used for making customized or premade dental jackets, full and partial dentures, full and partial implant dentures, fixed and removable implant bridges, implant crowns, Maryland bridges, surgical guides, milling blocks, and replacement teeth. In the present situation a unique composition has been developed for making the appliances

Typically, the manufacturing process begins with a preliminary impression of the patient's mouth, which is usually done in wax. This impression is used to prepare a diagnostic cast. While making the impression, the dentist applies pressure to the soft tissues to simulate biting force and extends the borders of the mold to adjacent toothless areas to allow the dentures to better adapt to the gums.

Once an appropriate preliminary cast has been obtained, the final cast is cast from, for example, gypsum, a stone-like product. The final mold is inspected and approved before using it to manufacture the teeth.

After the mold has been cast, it is filled with acrylic resin (in the present inventive embodiment the mold is filled with a unique polyurethane elastomer) to form the denture. The mold is prepared with a release agent prior to adding the resin to ensure that the hardened acrylic can be easily removed once the process is completed. A sheet of separating film between the acrylic and the model is also helpful in this regard. The denturist then mixes the appropriate resin compounds in liquid form. Upon drying, the resin hardens to a durable finish.

This resin mixture is packed into the mold. This method is more prone to air bubbles than hand packing.

Once the mold is packed to the denturist's satisfaction, it is heated to initiate the chemical reaction which causes the resin to harden.

After the heating is done and the mold has cooled, the mold is broken apart so the denture may be removed.

The denture or jacket is then put in the model of the patient's mouth to ensure that it fits and that the bite is good. Because of the number of processing steps there may be a slight discrepancy in the fit. Usually just a minor grinding and smoothing of surfaces is all that is necessary to make the denture fit correctly.

At this point, if the denture is the removable type, it is ready for use.

Implants require additional preparatory steps before they can be used. In one procedure, the denturist must drill the appropriate holes in the jaw bone and attach an anchor. After three to six months, when the hole has healed and the anchor is set in place, a small second surgical procedure is necessary to expose the implant and connect a metal rod to it that will be used to hold the crown or bridge. Finally, the replacement tooth is attached to the rod, where it is held firmly in place.

In appropriate cases, the denturist may extract teeth, install implant anchors, and attach a temporary appliance all in one office appointment. These new procedures have been called “all-in-one day” procedures. Some procedures are done to prepare the patient to accept a full implant bridge, a partial implant bridge, or individual implanted teeth.

Whatever the procedure used, the present inventive synthetic resin-based dental material may be used to make the dental appliance. The dental material results in a prosthesis which is able to withstand the forces of mastication and chewing by providing the prosthesis with the ability to absorb shock and stresses without cracking or breaking. In the present inventive process, the manufacturing process begins with a preliminary impression of the patient's mouth which is usually done in an Alginate impression. This impression is used to prepare a working cast. While making the impression, the dentist applies pressure to the soft tissues to simulate biting force and extends the borders of the mold to adjacent toothless areas to allow the appliance to better adapt to the gums. At this step, the clinician may also collect other useful information including bite registration, opposing model, smile design photos, etc.

Once an appropriate preliminary cast has been obtained, the final prototype cast is completed for the manufacture the teeth. Items needed include:
1). Polyvinyl working impression;
2). Matrix for final appliance “mock up;”
3). Components of the polyurethane elastomer and mixing equipment; and
4). Convection oven.

Pre heat oven to 200 F. This will help the matrix become hot faster.

Use a convection type oven in which the temperature is adjustable up to at least 200 degrees F. with a built-in 120 minute timer for worry-free baking.

Steam clean matrix and polyvinyl cast (PV); air dry to remove any moisture.

Place matrix and PV cast into oven and heat for one hour.

Remove items from oven, mix elastomer components into one uniform color. Mix material carefully as not to introduce air bubbles. Pour material into matrix of the appliance; place PV cast onto the matrix until properly seated. Let it sit for one to two minutes to keep from distorting the appliance; and place the whole thing into the oven.

Bake at 200 F. for one hour.

Remove from oven and place into the freezer for approximately 30 minutes.

Remove from freezer and de-mold. Do not tear the matrix in case it needs to be reused. Again, the style of oven to use should have the ability to reach 200 F and above and incorporate a timer for best results.

In the above discussion of the manufacturing process, the step of filling the mold is an important step. The present inventive method utilizes an injection gun which allows the technician to have side-by-side cartridges containing the components of the present inventive elastomer.

One cartridge contains a composition of a polyether polyol or polyester polyol with a functionality greater than two and which has a molecular weight greater than one hundred. One such composition is glycerol polypropylene glycol triether. The other cartridge contains a composition of an isocyanate prepolymer prepared from one or more branched polyester or polyether polyols with a functionality greater than two. The isocyanate may be either aliphatic or aromatic. One such composition is dicyclohexylmethane 4,4′-diisocyanate. Nanoparticles, as described below, may be dispersed in either of the components.

Application of a single action plunger will mix the components in a single stream in the proper proportions for use in filling the mold. Alternatively, each component may be initially in a “dry” form and converted to a liquid form as is known in the art. Each component may be mixed separately and then blended into one uniform liquid mixture for filling the mold. Colorants may be mixed into the polyurethane mixture to produce a shading to the composition.

As previously stated, the composition of the dental jacket uses principally a two-component, solvent free, nanoresin composite polyurethane system. The polyurethane system incorporates a polyol isocyanate, and nanoparticles.

Colorants may also be mixed with the composition. The nanoparticles used may be of various sizes, makeup, and density. The nanoparticles may be, but not limited to, barium glass, strontium glass, and aluminum oxide. The varying particle size of the nanos may be between 0.5 to 15 micrometers.

The composition of the polyol blend consists of a polyester with functionality greater than two and, a molecular weight greater than one hundred. The polyol blend that reacts with an isocyanate prepolymer prepared from one or more branched polyester or polyether polyols. The hydroxyl weight of the polyols may range from 56 to 400, preferably 200 to 600.

The polyether polyols may consist of polyfunctional alcohols such as glycerine, trimethylolpropane, pentaerythritol or polytetramethylene glycol. They may be polyether diols or triols with either ethylene oxide or propylene oxide end caps. The polyester polyols may be, but not limited to, dibasic acid reacted with a diol such as ethylene glycol, 1,2-propylene glycol, or diethylene glycol.

The polyol blend may be also composed of additives such as surfactants, moisture scavengers, antifoam additives, pigments, and stabilizers. Pigments or pigment dispersions of multiple colors may be used. They may be composed of, but not limited to, titanium dioxide, calcium carbonate, iron oxide, and the like dispersed in the polyether polyol.

The isocyanate may be either aliphatic or aromatic. Aromatic polyisocyanates may be, but not limited to, 2,4-tolune diisocyanate and 4,4′-diisocyanate. The aliphatic polyisocyanates may be, but not limited to, a hexamethylene diisocyanate, dicyclohexylmethane, 4,4′-diisocyanate, or isophorone diisocyanate. The isocyanate terminated prepolymer may be formed by reacting polyols with isocyanate with an excess of isocyanate. Functionality of the prepolymer itself should be greater than two.

This final composite structure is a reaction of the prepolymer with a polyol mixture in a volume or weight ration of the prepolymer and polyol blend of 1:1; and, therefore, is a polyurethane system providing a Shore D hardness of greater than 65.

It is to be understood that the invention is not to be limited to the exact details of operation or structure described as obvious modifications and equivalents will be apparent to one skilled in the art. The dental appliances herein can also be prepared employing any other known and conventional techniques known in the art.

We claim:

1. A dental appliance made of a hard, solvent free, substantially non-hydrophilic polyurethane, elastomer having a hardness of not less than about Shore D65 comprising:
a polyester or polyether polyol with a functionality greater than two and a molecular weight greater than one hundred reacted with an isocyanate prepolymer prepared from one or more branched polyester or polyether polyols with a functionality greater than two, said prepolymer and first polyester or polyether polyol blended with nanoparticles.

2. The dental appliance of claim 1 wherein said isocyanate prepolymer is aliphatic.

3. The dental appliance of claim 1 wherein said isocyanate prepolymer is aromatic.

4. The dental appliance of claim 1 wherein said polyester or polyether polyol comprises glycerol polypropylene glycol triether and said isocyanate prepolymer comprises dicyclohexylmethane 4,4′-diisocyanate.

5. The dental appliance of claim 1 wherein said appliance is a dental jacket.

6. The dental appliance of claim 1 wherein said appliance is selected from the group consisting of a customized or premade dental jacket, a full or partial denture, a full or partial implant denture, a fixed or removable implant bridge, an implant crown, a Maryland bridge, a surgical guide, a dental milling block, and a replacement tooth.

7. The dental appliance of claim 2 wherein said appliance is selected from the group consisting of a customized or premade dental jacket, a full or partial denture, a full or partial implant denture, a fixed or removable implant bridge, an implant crown, a Maryland bridge, a surgical guide, a milling block, and a replacement tooth.

8. The dental appliance of claim 4 wherein said appliance is selected from the group consisting of a customized or premade dental jacket, a full or partial denture, a full or partial implant denture, a fixed or removable implant bridge, an implant crown, a Maryland bridge, a surgical guide, a milling block, and a replacement tooth.

9. A synthetic resin-based dental jacket made of a composition comprising:
a hard, solvent free, substantially hydrophilic polyurethane elastomer incorporating nanoparticles, said elastomer having a hardness of not less than about Shore D65, said hard elastomer consisting essentially of the reaction product of a polyester or polyether polyol with a functionality greater than two and a molecular weight greater than one hundred and an isocyanate prepolymer prepared from one or more branched polyester or polyether polyols, said prepolymer with a functionality greater than two, said isocyanate being either aliphatic or aromatic.

10. The synthetic resin-based dental jacket of claim 9 wherein said polyester or polyether polyol comprises glycerol polypropylene glycol triether and said isocyanate prepolymer comprises dicyclohexylmethane 4,4′-diisocyanate.
